(19) United States
(12) Patent Application Publication
(10) Pub. No.: US 2013/0338598 A1
(43) Pub. Date: Dec. 19, 2013

(54) Removal of Cannula Part from Base Part

(71) Applicant: Steffen Gyrn, Ringsted (DK)

(72) Inventor: Steffen Gyrn, Ringsted (DK)

(73) Assignee: Unomedical A/S, Birkerod (DK)

(21) Appl. No.: 13/983,394

(22) PCT Filed: Feb. 7, 2012
(86) PCT No.: PCT/EP12/52037

§ 371 Date: Aug. 27, 2013

Related U.S. Application Data
(60) Provisional application No. 61/441,038, filed on Feb. 9, 2011.

(30) Foreign Application Priority Data
Feb. 10, 2011 (EP) 11154049.8

(51) Int. Cl. A61M 5/158

(52) U.S. Cl. CPC A61M 5/158
USPC 604/174

(57) ABSTRACT

The invention concerns a base part system for a medication delivery device. The base part system to be combined with a detachable reservoir/delivery part according to the application comprises a plate (1) provided with a mounting surface (1a) and openings (12a, 12b) through which two or more subcutaneous units in the form of at least two subcutaneously positioned cannulas (22a, 22b) or at least one subcutaneously positioned cannula and e.g. a subcutaneously positioned part of a sensor extend during use. During use at least one subcutaneous unit in form of a cannula part is attached to the plate (1) with fastening means (25). The subcutaneous unit comprises a body part (7) placed above the patient's skin which is provided with handling means (26) allowing a user to detach the subcutaneous unit from the plate (1) and to remove the used subcutaneous unit from the patient.

### TECHNICAL FIELD OF THE INVENTION

The invention concerns a base part system for a medication delivery device. The system comprises a base part plate and two or more units positioned subcutaneously during use, at least two of the subcutaneously positioned units are cannula parts. The two subcutaneously positioned units might be subcutaneously positioned in overlapping time intervals or in time intervals which are not overlapping. The base part plate is during use fastened to a patient's skin and connected to a separate cannula part which cannula part is positioned at least partly subcutaneous. At least one second and e.g. several other cannulas is/are also connected to the base part plate. Handling means are connected to the cannula part whereby the cannula part can be easily retracted after a period of use.

### BACKGROUND OF THE INVENTION

The document US 2004/0162521 discloses a needle device comprising a housing, a base portion having a mounting surface adapted for application to the skin of a patient and a plurality of needles. Each needle comprises a distal pointed end adapted to penetrate the skin of a patient and each needle has a first position in which the distal end is retracted relative to the mounting surface and a second position in which the distal end projects from the mounting surface. A needle device according to this document being mounted on the patients has to have a height at least corresponding to the length of a needle as the needles before and after use are retracted in their full length perpendicular to the mounting surface, also the cannulas according to the shown embodiments have to be hard, self-penetrating cannulas provided with a side inlet opening.

The document US 2008/0004515 discloses an integrated analyte monitoring system combined with an on-body patch pump provided with multiple cannulas and a sensor combination. In accordance with an embodiment of this document a first cannula can be configured for transcutaneous delivery of a medication at a first infusion site for an initial time period of e.g. three to four days. Thereafter the first cannula is retracted from the infusion site under the control and operation of one or more controller and infusion management units. After retraction of the first cannula, a second cannula can be inserted at a second infusion site. The second cannula may be inserted automatically by using an insertion device such as an insertion gun configured to couple to the second cannula e.g. including a spring bias driven insertion mechanism. The second cannula (290) is mounted on a base part separate from the patch pump (210) in connection with which the first cannula is mounted.

Neither of the devices mentioned in the above prior art documents allows for retraction of a cannula without removing the patch which the cannula(s) are part of and does not describe the use of handling means connected to the cannula part to ease or to render possible the retraction of the cannula after a period of use without retraction of the base part.

US 2008/0199707 relates to a medical device for monitoring analytes in a living body and delivering fluids thereto, such as monitoring glucose levels and delivering insulin to people with diabetes. More particularly, the invention relates to analyte monitoring and fluid delivery systems integrated into a flexible patch. In some embodiments conductive pathways are formed in the fabric of the patch. Components that may be integrated with the flexible patch include: a power source, controller, transmitter, antenna, temperature and other sensors, fluid pump, infusion set, electrical pathways, switches, control electrodes connectors, resistors and other circuit elements. Such components may be embedded, interwoven or coated on to the flexible patch instead of or in addition to surface mounting. The flexible patch can be constructed of polyester, nylon, polyurethane, Lycra® or other synthetic or natural fibers. The patch may have elastomeric properties that come from properties of the fibers themselves or from how the fibers are combined to form the patch. The flexible patch may be woven, non-woven, knitted, spun or constructed of a textured film.

The flexible patch may be provided with one or more sensor sites, the sensors may include adhesive mounts or some type of mounting feature such as one or more snaps, hooks, clamps, pins, clips or other means molded onto or attached to the patch to secure the sensor to the flexible patch or the users skin during use.

The flexible patch may also be provided with an infusion set (24) which infusion set may include an adhesive mount (46) for securing the distal end of infusion set (24) to the patch (12) or directly to the patient's skin. Multiple sites (50) may be provided in the patch (12) for alternately placing infusion sets (24). After a predetermined period of use, typically 3 days, infusion set (24) can be removed by lifting adhesive mount (46), removing cannula (44) from the patient and disconnecting tubing (42) from output port of the reservoir. A fresh infusion set (24) may then be placed in another one of the sites (50) and connected to the reservoir.

The infusion set according to US 2008/0199707 does not as such comprise any handling means as the infusion sets are attached by adhesion which adhesion assumably only causes a light adherence between the infusion set and the patch and therefore it would be possible to “lift” adhesive mount (46) and remove the cannula (44). It would be very difficult for a user to actually handle a device according to the applicants given directions.

### SUMMARY OF THE INVENTION

The object of the invention is to provide a base part system to be combined with a detachable reservoir/delivery part, the base part system comprising:
a plate provided with a lower mounting surface and openings through which two or more subcutaneous units in the form of at least two subcutaneously positioned cannulas and e.g. a subcutaneously positioned part of a sensor extend during use,
during use at least one subcutaneous unit in form of a cannula part is attached to the plate with fastening means, said at least one subcutaneous unit comprises a body part placed above the patient's skin and being provided with an inner cavity and with an inlet opening through which fluid can enter the inner cavity and an outlet opening through which the fluid can exit the inner cavity and enter an at least partly subcutaneously positioned cannula.

The body part of the at least one subcutaneous unit is provided with handling means, said handling means allowing a user to detach the subcutaneous unit from the plate and remove the used subcutaneous unit from the patient.

The presence of the hard-walled body part of the cannula part allows for establishing different fluid paths from the reservoir to the cannula. Especially, the hard-walled body part allows for establishing rigid fluid paths made of molded rigid plastic or metal making it possible to avoid the use of soft tubes in order to establish a fluid path from the reservoir to the cannula. It is desirable to avoid the soft tube connections as the fluid path between the reservoir and the cannula should either be very short or alternatively, it should be replaced when replacing the cannula in order to avoid having dead volumes of possibly contaminated medication standing in the fluid path. When replacing a soft tube connection the user will have to establish the new connection when replacing the cannula part, considering the very small dimensions of the whole base part system, it will be very difficult to provide a solution which will make it easy or even possible for a user to attach the end of a soft tube to a reservoir unit. When dealing with rigid fluid paths, it will be possible to provide solutions where the user will be able to position larger units relative to each other instead of positioning the tiny end of a soft tube relative to the reservoir unit. E.g. according to the embodiment of FIG. 1A-E, the user has to reposition the whole reservoir unit relative to the base plate in order to change the fluid path to the cannula part and in the embodiment of FIG. 2 the user has to slide a rigid movable part from a first to a second position in order to change the fluid path.

According to one embodiment the plate is made of a relatively rigid material having a Young's Modulus >0.6 GPa.

According to one embodiment the inlet opening to the inner cavity is either covered and closed by a septum or encircled by a sealing or gasket providing a fluid tight closure to a fluid supply thereby preventing access of microorganism to the inner cavity after a cannula part has been inserted into the plate. The sealing/gasket can either comprise two sealing parts where the first sealing part encircles the cannula part below the inlet opening and the second sealing part encircles the cannula part above the inlet opening, or the sealing/gasket can comprise a single sealing encircling the inlet opening.

Normally, the handling means comprises other part than the fastening means i.e. the handling means are different from the fastening means, and according to one embodiment the handling parts are not in direct contact with the fastening means but are positioned above i.e. in a further distance from plate than the fastening means. This means that the user can place the finger tips below or beside the handling means while the fastening means are in engagement with the plate, and in this situation where the handle means are handled the fastening means will normally be placed below the finger tips. “Below” indicates closer to the patient's skin, the “lowest” surface being the patient's skin.

The two subcutaneously positioned units might be subcutaneously positioned in overlapping time intervals or in time intervals which are not overlapping. This means that e.g. two subcutaneous units can be inserted at day 0 and then one unit is removed after 3 days, or e.g. a first subcutaneous unit is inserted day 0, and after 3 days a second subcutaneous unit is inserted and the first unit removed. The first unit might in this situation be removed either before or after insertion of the second unit. The use time of the base part system might be extended by further 3 day be inserting a third subcutaneous unit.

According to one embodiment said at least one subcutaneous unit before use is releasably attached to an inserter and during insertion said unit is attached to the plate with fastening means.

According to one embodiment said fastening means is a non-hardening adhesive agent allowing said subcutaneous unit to attach and to detach from the plate.

According to one embodiment said fastening means (25) comprise pivotal hooks having a locked and an unlocked position relative to the plate (1), allowing said subcutaneous unit to attach and to detach from the plate (1).

According to one embodiment said fastening means (25) are positioned at the lower side of said body part (7) of said at least one subcutaneous unit.

According to one embodiment the part placed subcutaneously (24) is a soft, non-penetrating cannula.

According to one embodiment said handling means (26) attached to said body part of said at least one subcutaneous unit is provided with a surface facing the patients skin thereby providing a contact surface making it possible for the user to affect the subcutaneous unit with a force in a direction opposite (180°) the direction in which the part placed subcutaneously (24) is inserted into the patient and being large enough to overcome the force attaching the subcutaneous unit to the plate or the patients skin.

According to one embodiment said handling means (26) attached to said body part of said at least one subcutaneous unit is provided with a surface positioned perpendicular to the patients skin making it possible to first affect the subcutaneous unit with a force directed parallel to the patient's skin and being large enough to overcome the force keeping the fastening means (25) of the subcutaneous unit in an attached position in the plate (1) whereby the fastening means 25 are released and second affect the subcutaneous unit with a force in a direction opposite (180°) the direction in which the part placed subcutaneously (24) is inserted into the patient and being large enough to overcome the force attaching the subcutaneous unit to the plate or the patient's skin.

Also, the invention relates to a subcutaneous unit comprising a body part (7) placed above the patient's skin, a part placed subcutaneously (24) and fastening means (25) for attaching the subcutaneous unit to a base plate (1), wherein said subcutaneous unit is provided with handling means (26) attached to said body part, said handling means allowing a user to detach the subcutaneous unit from a part of the plate (1) and dispose of the used unit.

According to one embodiment the subcutaneously placed part (24) of the subcutaneous unit is a soft, non-penetrating cannula.

According to one embodiment said handling means (26) comprises hooks, whereby said subcutaneous unit can be detached from a plate (1) and removed from a patient by pressing said hooks towards each other.

### DEFINITIONS

“Parallel” or “essentially parallel” as used herein refers to a second movement in a direction, plane, item or the like defined in relation to a first or a reference plane or direction which reference plane or direction has a direction defined as the angle a=0°; and the second plane or direction deviates at maximum ±100; normally not more than ±5° from the first or reference direction a.

In the context of the application “horizontal” or “essentially horizontal” means that a movement in a direction, a direction, plane, item or the like is horizontal or essentially horizontal is parallel or essentially parallel to the surface of the skin of a patient as defined above. For example, the base part to which the insertion device is fastened can be horizontal, or essentially horizontal, parallel or essentially parallel to the skin.

“Perpendicular” or “essentially perpendicular” as used herein refers to a second movement in a direction, a direction, plane, item or the like defined in relation to a reference plane or direction which reference plane or direction has a position or a direction in the angle ˜=0°; and the second plane or direction deviates between 80-100°; normally between 85-95° from the first reference ˜.

In the context of the application “Transversal” or “essentially transversal” can be used interchangeably with perpendicular or essentially perpendicular as defined above.

“Means”: As used herein, the expression “means” can comprise one or more means. This is irrespective, if with respect to grammar, the verb relating to said means indicates singular or plural.

### BRIEF DESCRIPTION OF THE DRAWINGS

A detailed description of embodiments of the current invention will be made with reference to the accompanying figures, wherein like numerals designate corresponding parts in different figures.

FIG. 1A-E shows an embodiment of a base part having two separated positions for cannulas and a delivery part mounted in each of these two positions.

FIG. 2 shows an embodiment of a base part where the fluid path is established by pushing a common part.

FIG. 3A-B show an embodiment of a base part where the cannula through which medication is lead to the patient can be removed after use while the main part of the base part remains in position on the patient's skin. FIG. 3C shows an enlargement of a stationary connector part.

FIG. 4A-B show a side view of one embodiment of the fastening means and how this fastening means can be removed whereby the cannula part can also be removed from the user.

FIG. 5 shows an embodiment of a base plate with releasable sites for a cannula.

### DETAILED DESCRIPTION OF THE INVENTION

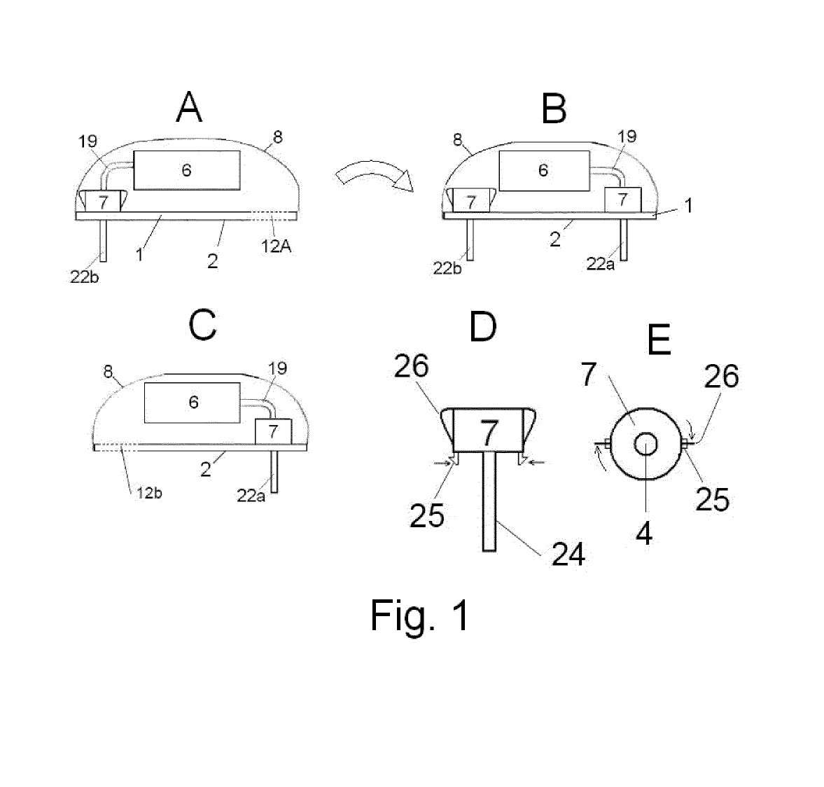

FIG. 1A-E shows an embodiment of a base part system comprising a plate 1 with two separate openings 12a, 12b through which openings subcutaneous units in the form of cannula parts 22a, 22b can be mounted. The plate 1 is normally made of a hard and relatively rigid material such as a metal or hard plastic e.g. polyethylene (PE), polypropylene (PP), polystyrene (PS) or the like.

Often, the definition “relatively rigid” is used as the rigidity of the material will depend not only of the chosen material but also of the dimensions of the material such as thickness and width of the plate, i.e. the same material can be made relatively more rigid by increasing the thickness of the plate.

Normally, a material will be considered rigid in this technical area if it has a Young's Modulus >0.6 GPa (approximately 87.000 lbf/in² (psi)). That a plate according to the claims is mainly made of a hard material means that a minor part of the plate i.e. less than ⅓, normally less than 20%, of the plate area, can consist of a softer, more flexible material. E.g. one or both sides i.e. upper or lower side or parts of one or both sides of the plate can be covered with a softer material adding other properties to the plate, e.g. making it easier to fasten units to the upper side or to provide an adhesive surface on the lower side i.e. the side turned toward a patient's skin during use. Also, the plate 1 could be provided with areas e.g. lines of thin or otherwise weakened material which would make it possible for the plate 1 to bend along such a line.

When the plate 1 is made of a relatively rigid material it is easy to provide different positions where a user of the device can position exchangeable parts such as reservoir and cannula part. If the plate 1 is made of a softer and more compliant material it could be more difficult to provide the surface of the plate 1 with guiding means providing a safe and intuitive positioning of the exchangeable parts. Also the use of a rigid material such as metal or plastic makes it possible to provide a rigid fluid path from the reservoir to the cannula part delivering medication to the patient. Use of such rigid material prevents closing of the fluid path resulting from reduction of the flow area as the walls of the flow path cannot be squeezed together thereby reducing the fluid flow through the pipe forming the fluid path.

Generally, a subcutaneous unit according to the application comprises a body part which during use is positioned above the patient's skin and a longish subcutaneously positioned part in form of a soft or self-penetrating cannula or a part of a sensor which is positioned at least partly subcutaneously, i.e. either subcutaneously in the hypodermis or intramuscularly.

FIG. 1 shows a subcutaneous unit in form of a cannula part comprises a body part 7 and a subcutaneously positioned part 24 in form of a soft or self-penetrating cannula which during use is inserted below the patients skin surface. During use, the cannula part is mounted through the opening 12a or 12b and each cannula part 22a, 22b can provide a fluid path from an inlet in the body part 7 to an outlet in the cannula 24, the inlet of the body part 7 is provided with a septum 4. The body part 7 of the cannula part comprises fastening means 25 and according to the embodiment shown in FIG. 1A-E, the fastening means is constituted by two oppositely positioned outward hooks which are pivotally fastened to the body part 7. During insertion each outward hook 25 will be pushed inward towards the centre of the body part 7 as the inclined surface of the hook will slide along an edge of the rigid plate 1 and this contact will force the pivotally fastened hooks inwards i.e. toward the centre of the body part 7 as illustrated with the arrows in FIG. 1D.

Generally, fastening means 25 are needed to secure the subcutaneous unit to the plate 1—or alternatively to the patients skin—when the subcutaneously positioned part is fully inserted into a working position. If the subcutaneous unit is not properly fastened, it is unlikely that the subcutaneously positioned part will remain in the position in which it is positioned by insertion. Alternatively, the fastening means can be a non-hardening adhesive agent allowing the subcutaneous unit to attach and to detach from the plate. Normally, the fastening means are positioned at the lower side of the body part 7 i.e. the side or surface which is facing the plate 1 or the patients skin during use.

Generally, a subcutaneous unit according to this application is provided with handling means 26 attached to the body part 7, the handling means allowing a user to take hold on the body part 7 and to detach a cannula part from the plate 1 either directly by grapping and pulling the used unit from the patient in a direction opposite the insertion direction or indirectly by first unlocking the cannula part e.g. by twisting or otherwise releasing the fastening means 25 and then pulling the used unit away from the patient in a direction opposite the insertion direction.

According to the embodiment shown in FIG. 1A-E, the handling means 26 have the form of oppositely positioned protruding parts attached to side surfaces of the body part 7 of the subcutaneous unit. These handling parts 26 make it possible to pivot the body part 7 of the cannula part around the central axis along the subcutaneously positioned part 24 to a position where the fastening means 25 i.e. the outward hooks are not locked by the plate 1 as openings in the inward edge of the opening in the plate 1 allows for the cannula part to be pulled away from the base part plate 1 at a given position.

Alternatively, the handling means 26 can have the form of a ring attached to the body part of the subcutaneous unit by which grapping and pulling the ring in a direction opposite the insertion direction of the subcutaneously positioned part will release the used unit from the plate 1 and the patient. Before pulling, it might be necessary to detach the subcutaneous unit by first unlocking the subcutaneous unit from the plate 1 e.g. by twisting or otherwise releasing the ring and then pulling the ring away from the patient in a direction opposite the insertion direction.

According to a yet an embodiment, the handling means 26 can have the form of a collar attached to the circumference of the body part 7 by which grapping and pulling the collar in a direction opposite the insertion direction of the cannula part will release the used unit from the patient. Also according to this embodiment, it might be necessary to detach the subcutaneous unit by first unlocking the subcutaneous unit e.g. by twisting or otherwise releasing the collar and then pulling the collar away from the patient in a direction opposite the insertion direction.

Any other shape of the handling means allowing the user to detach the subcutaneous unit from the plate either directly by grapping and pulling the used unit from the patient in a direction opposite the insertion direction or indirectly by first unlocking the cannula part e.g. by twisting or otherwise releasing the fastening means and then pulling the used unit away from the patient in a direction opposite the insertion direction, can be used according to the present invention.

By incorporation of handling means 26 in connection with the subcutaneous unit it is possible to completely retract the subcutaneously positioned part with the handling means from the plate 1 and thereby from the patient. Normally, a subcutaneously positioned part such as a cannula part has to be removed from a patient after approx. 3 days since otherwise the infection risks are too high and the intake of medication might no longer be optimal. The use of handling means 26 extent the lifetime of the main parts of the base part since it is not necessary to remove the whole plate 1 but only the one cannula part, where after a second cannula part can take over extending the lifetime of the device with approx. 3 days.

The plate 1 comprises a flat surface having a lower side, the lower side being placed next to the patients skin during use is provided with a mounting surface 2; normally the mounting surface will consist of a pressure adhesive layer either welded to the lower side of the flat surface of the plate 1 or adhered directly to the lower side of the plate. On the upper side of the plate are normally means for fastening a delivery part 8 in position during use. The delivery part 8 can either be ready to be mounted or be pre-mounted to the base part system. The delivery part 8 is normally detachable which means that the delivery part 8 can be fastened to the plate 1 and released again. On the upper side of the plate 1 are also fastening means corresponding to the fastening of the cannula part. Generally, a cannula part or another subcutaneous unit such as e.g. a sensor can before use be placed in an inserter i.e. the subcutaneous unit is releasably attached to the inserter and during insertion the subcutaneous unit is released from the inserter and attached to the plate 1 by use of the fastening means 25 of the body part of the subcutaneous unit and the corresponding fastening surface or means of the plate 1.

The delivery part 8 shown in FIG. 1A-C comprises a reservoir 6 having an outlet connected to a connector 19 in the form of a connector needle; fastening means for fastening the reservoir 6 are not shown in FIG. 1. The connector 19 provides a first fluid path of fluid from the reservoir to the patient and ends in a needle which is able to penetrate the septum 4 of the cannula part. A second fluid path is constituted by the open room inside the cannula housing as this open room permits transfer of fluid from the outlet of the first fluid path i.e. the connector 19 to the open end of the tube shaped cannula 22a, 22b which is embedded and secured to the inside of the body of the cannula part 7. The volume of this open room is considered to be so little that plug-flow is still obtained. Alternatively, a sprinkler cannula having more than one opening could be used.

The fluid path is changed when the position of the delivery part 8 is changed. In FIG. 1A the delivery part 8 is in a first state where the connector 19 of the delivery part 8 is penetrating a septum in the body part 7 of a first cannula part having a subcutaneous cannula 22b thereby providing a first and a second fluid path. In FIG. 1B the delivery part 8 is in a second state where the connector 19 of the delivery part 8 is penetrating a septum in a body part 7 of a second cannula part having a subcutaneous cannula 22a thereby providing a new first and second fluid path. The second state for the delivery part 8 is in this embodiment obtained by turning it 180° in a horizontal plane relative to the plate 1.

In FIG. 1C the first cannula part 7 having a subcutaneous cannula 22b has been removed from the base part system.

Generally, removal of the cannula part is achieved by affecting the body part 7 of the subcutaneous unit with a force directed away from the patient's skin which force is large enough to overcome the force attaching the subcutaneous unit to the plate 1 or the patient's skin. This can be obtained by providing the body part 7 with a surface facing the patients skin which surface can be touched or otherwise influenced by the users fingers, thereby making it possible to affect the body part 7 with a force directed away from the patients skin and being large enough to overcome the force attaching the subcutaneous unit to the plate 1 or the patient's skin. The surface of the body part 7 facing the patient's skin is normally placed in an angle parallel to the patient skin or at least in an angle ±45°. This feature allows the subcutaneous unit to be carefully maneuvered by grapping and pulling the surface that is facing the patient's skin in a direction opposite (180°) or essentially opposite the direction in which the cannula is inserted into the patient. This way, the subcutaneous unit is detached from the plate and removed from the patient by pulling in a direction away from the patient's skin. The subcutaneous unit is detached from the plate to such a degree that the used unit and maybe also a detachable part of the plate to which the subcutaneous unit is originally attached is completely removed from the base part system and disposed off where after the risk of getting in contact with the used cannula is completely eliminated.

Alternatively, the handling means 26 attached to the body part 7 of the at least one subcutaneous unit is provided with a surface positioned perpendicular or essentially perpendicular to the patients skin making it possible to first affect the subcutaneous unit with a force directed parallel to the patients skin and being large enough to overcome the force keeping the fastening means 25 of the subcutaneous unit in an attached position in the plate 1 or to simply shift the fastening means 25 to an unlocked position whereby the fastening means 25 are/can be released and secondly affect the subcutaneous unit with a force directed away from the patients skin and being large enough to overcome the force attaching the subcutaneous unit to the plate or the patient's skin. This feature allows the subcutaneous unit to be carefully detached from the plate and remove the used unit from the patient by first turning or twisting and then pulling away.

It is desirable that the cannula is removed from the patient in a direction opposite (180° or essentially opposite the direction in which the cannula is inserted into the patient, since otherwise the patient might experience discomfort.

The base part system according to the present invention can e.g. deliver insulin based on a measurement of glucose in the patient's blood.

The device according to the present application is especially directed towards use of a subgroup of cannulas known as soft needle cannulas and they have a wide range of applications, e.g. in automated drug delivery devices such as insulin delivery devices. The soft needle cannulas are in general more flexible and softer than other cannulas. The soft needle cannulas are used together with an introducer needle (not shown in the figures), where the introducer needle is used to penetrate the barrier to the body e.g. the skin and assist the introduction of the cannula. The needle is removed after introduction of the cannula into a body cavity. The soft needle cannula is left in the body cavity for a desired period of time in which it functions as the means for drug delivery. A soft cannula can e.g. be made of a polymer that is more comfortable than a self-penetrating cannula made of e.g. metal or a hard polymer, but a soft cannula needs to be positioned subcutaneously with an inserter providing a separate penetrating needle able to cut through the patient's skin.

A soft needle cannula comprises a tube-shaped flexible part and a body part. The tube-shaped flexible part is adapted for insertion into a patient and it facilitates the fluid transport to or from a body cavity. The tube-shaped part must be flexible in order to allow the carrier of the cannula, e.g. a patient, to move without serious unpleasantness. However, it must not be so flexible that it is likely of forming kinks which may stop the drug delivery. The body part is the connecting means on the tube shaped part adapted for connecting the tube shaped part to either the drug delivery devise, to the fluid collecting container or to another connecting means e.g. a second tube. Preferably, soft needle cannulas are composed of a material which are sufficiently flexible to bend, when the carrier moves and sufficiently rigid to avoid kinking closing off the drug supply. Further the material must be compatible with medical use i.e. irritation of the skin must be kept at a minimum, being non-toxic it must not decompose in the body, etc. Thermoplastic elastomers (TPE) are a type of material which fulfils these requirements. Examples of such useful elastomers are: polyester ethers, ECOEL, styrene based TPE, olefin based TPE, urethane based TPE, ester based TPE, amid based TPE, polyolefines and silicone rubbers. In a preferred embodiment the material is selected from the group consisting of polypropylene, C-FLEX™, mixtures of C-FLEX™ and polypropylene, LUPOLEN™ 1840H, LUPOLEN™ 30200, PELLETHANE™ 2363-750, PELLETHANE™ 2363-550, TECOTHANE™ and CARBOTHANE™

Alternatively, the cannula part can have a side opening as inlet instead of a top opening as shown in the figures. Then the body part is provided with a protruding front having a flat surface provided with an opening. The protruding front of the cannula part need not be flat; it can actually have any desired shape as long as it is possible to create a corresponding surface on the connection part facing the cannula part. The front can be inclined in such a way that the cross-section at the upper i.e. distal end of the cannula part is larger than the cross-section at the proximal end of the front, i.e. the end closest to the patient after insertion. The opening of the protruding front is an inlet or outlet through which liquid can enter or exit the cannula part. The body is further provided with a top opening which can be covered with a self closing membrane i.e. a septum. The top opening need some kind of entrance protection as it is facing an outer surface which is in contact with the surroundings.

The top opening is primarily used when inserting the cannula part if the cannula 22 is a soft cannula which cannot by itself penetrate the patients skin, in this case it is necessary to use a pointy insertion needle of a relatively hard material when inserting the cannula and this pointy needle can be inserted through the top opening, pass through an inner hollow in the body of the cannula part and further pass through the full length of the cannula in such a way that the pointy end of the insertion needle stick out of the open end of the hollow cannula. After insertion i.e. after the cannula has been placed sub- or transcutaneous in the patient, then the insertion needle is retracted and the cannula is left inside the patient.

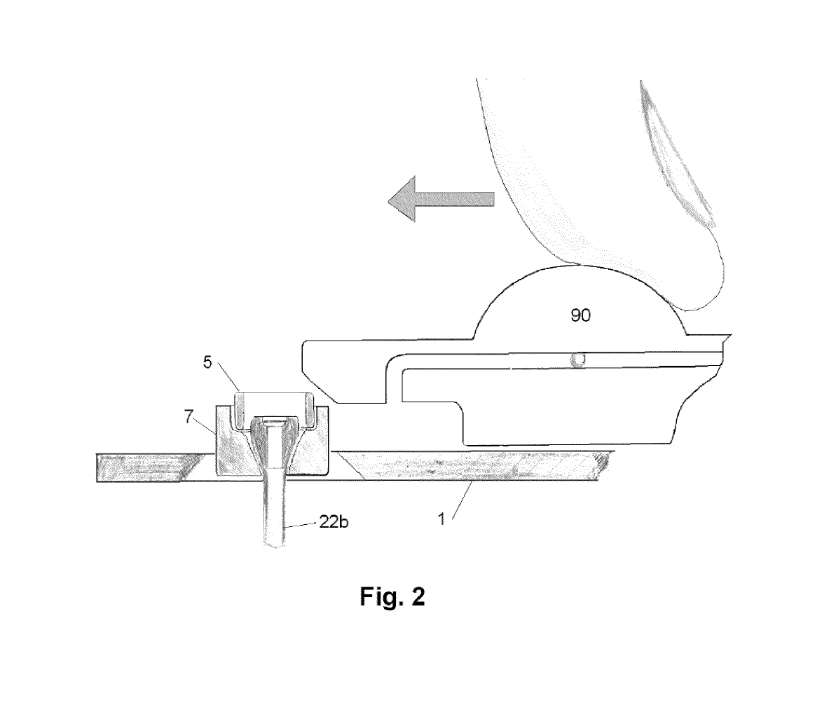

FIG. 2 shows an embodiment of a base part plate 1 where a fluid path is established by pushing a moving part 90 between a first and a second position. The moving part 90 is an unreleasable part of the base part plate 1 i.e. it cannot be removed from the base part but only be moved in a sliding way between different positions e.g. more than two positions.

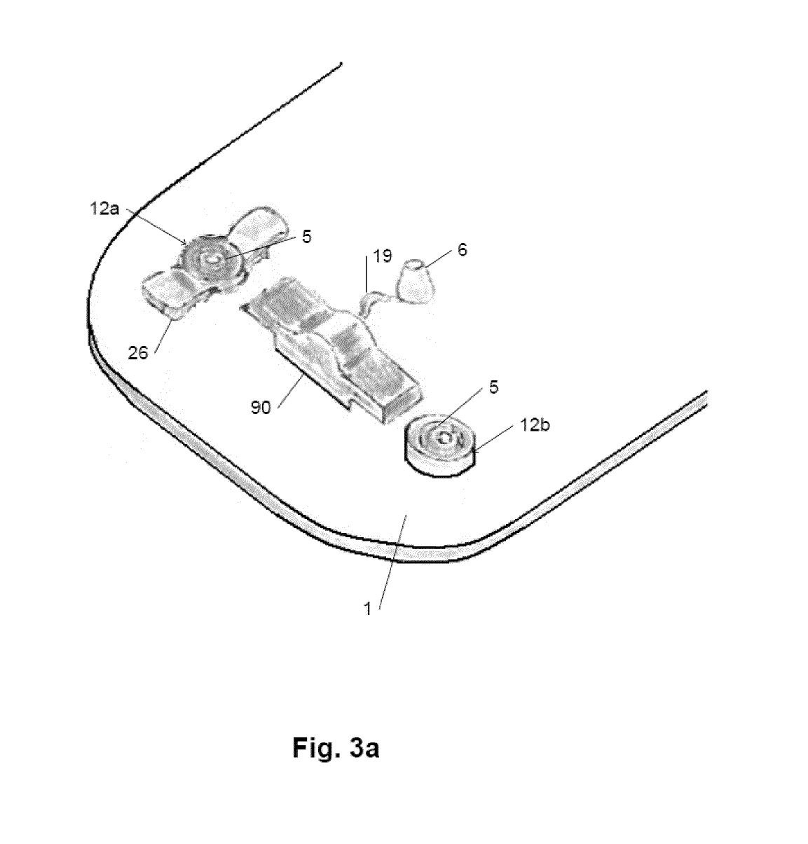

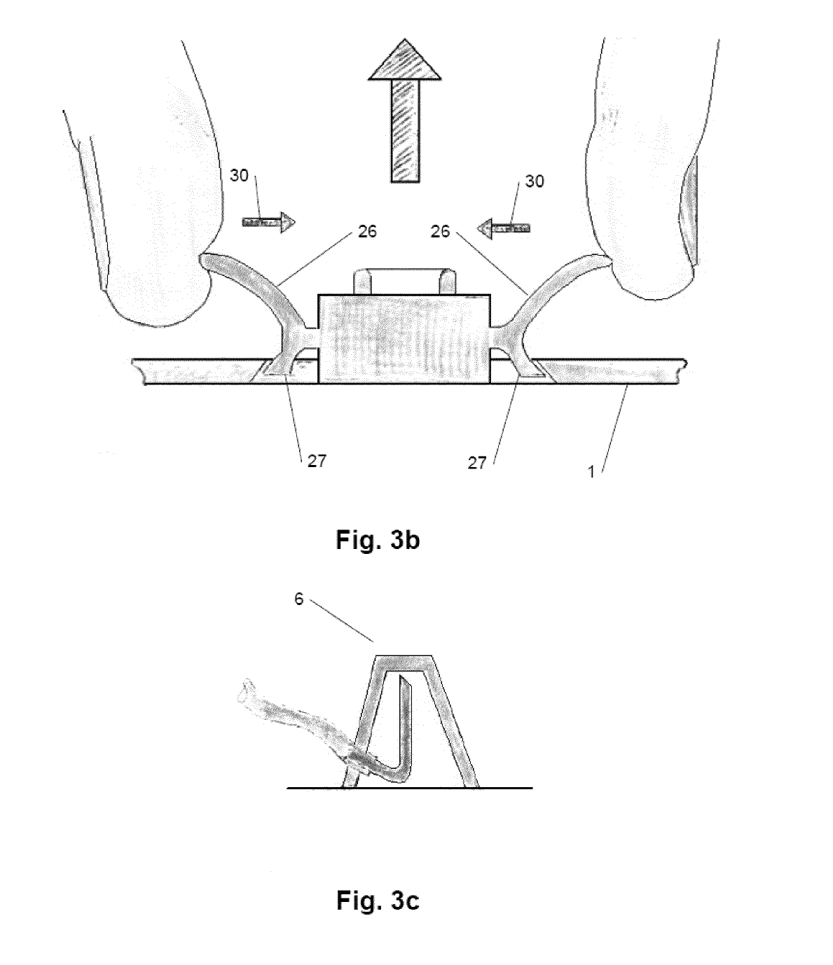

The moving part 90 has two outlets for fluid and through the first outlet fluid can be delivered to an inlet in a first cannula part guiding fluid to the first cannula 22b. Through the second outlet fluid can be delivered to an inlet in a second cannula part guiding fluid to the second cannula. The inlet for fluid in the each cannula is not protected by a septum but is constituted of an opening surrounded by a sealing or gasket 5 which allows flow of fluid from the moving part to the cannula part but prevents fluid escaping between the two units. In FIG. 3A the movable part 90 is in a neutral position where the fluid cannot flow from the connector 19 via the movable part 90 to the cannula.

The movable part establish the fluid path between a reservoir and a cannula, and by changing the position of the movable part the direction of the fluid path is changed from a first cannula to a second cannula.

After a period of approximately 3 days, the fluid path to the first cannula is stopped by moving the movable part to a neutral position and then the first cannula part is retracted by manually grapping and pulling the handling means as described above.

Then the second cannula, which either can be inserted at this point in time or can have been kept under sterile conditions inside the base part, is inserted, thereby making it possible to use the patch for another 2-3 days. The fluid path to the second cannula part is established by moving the movable part towards the second cannula part.

By incorporation of handling means in connection with the cannula part as described above it is possible to completely retract the cannula part from the base part plate and thereby from the user. The cannula part has to be removed from the user after approx. 3 days since otherwise the infection risks are too high. The use of handling means extent the lifetime of the device since it is not necessary to remove the whole device but only the one cannula part, where after a second cannula part can take over extending the lifetime of the device approx. 3 days.

FIGS. 3A, B and C shows an embodiment of a base part system provided with two cannula parts. FIG. 3a shows a plate 1 with two openings 12a, 12b for cannula parts in the base part plate, each cannula part is mounted through an opening in the base part plate and the inlet of the cannula part is provided with a gasket. The plate 1 comprises a flat surface having a lower side, the lower side placed next to the patients skin during use, is provided with a mounting surface; normally the mounting surface will consist of a pressure adhesive layer either welded to the lower side of the base part plate or adhered directly to the lower side of the base part plate. The embodiment shown in FIG. 3a has on the upper side of the base part plate a moving part 90 which is unreleasably fastened to the base part plate but can move relative to the upper surface, and a connector 19 for a not shown reservoir. The object of having two openings for cannulas is to be able to retract one cannula while inserting another cannula and still be using the same base part place or at least the main part of the same base part plate and e.g. also the single sensor which might be attached to the base part plate at a not shown position. Providing one or more of the earliest inserted cannula parts with handling means, will increase the service life of a base part plate as a cannula normally will have to be retracted after 3 days while the rest of the device normally can stay inserted in 6-10 days. After a period of e.g. 3 days, a first cannula is retracted and a second cannula is inserted, thereby making it possible to use the patch for another 2-3 days.

A connector 19 in the form of a flexible tube forms part of a non-rigid connection between the movable part and a not shown reservoir thereby establishing a fluid path from the reservoir to the patient. This connector 19 is an integral part of the plate 1 and is not in contact with the exchangeable cannula part. The connector 19 ends in a connector needle for the reservoir which connector needle is protected by a bubble shaped membrane (see enlargement in FIG. 3C). Hereby a first fluid path between the delivery device and an inlet on the base part can be established providing transfer of medication e.g. insulin, other medication or nutrients from the reservoir to the base part via the connector 19 and the moving part 90.

The movable part 90 establish the fluid path between the reservoir and the cannula, and by changing the position of the movable part the direction of the fluid path is changed from the first cannula to the second cannula.

In general, after the period of e.g. 3 days, the fluid path to the first cannula is stopped by moving the movable part 90 to a neutral position and then the first cannula part is retracted by manually grapping and pulling the handling means 26 as described above. Then the second cannula, which either is inserted, thereby making it possible using the patch for another 2-3 days. The fluid path to the second cannula part is established by moving the movable part 90 towards the second cannula part.

FIG. 3b shows a side view of the handling means 26 and how this handling means can be grabbed whereby the cannula part is also removed from the user. Generally, the fastening means secures the cannula part to the plate when the subcutaneous part—in this case a cannula—is fully inserted into a working position. In this embodiment the fastening means are embodied as hooks 27 ensuring a tight connection to the base part plate 1. When the user wants to detach the cannula part from the base part plate, the user manually presses the handling means 26 towards each other, illustrated in the figure by the fingers and the arrows 30 thereby releasing the tight fit between the hooks 27 and the base part plate 1. This enables that the handling means 26 can be removed and the cannula part follows.

The fastening means 25 secure the cannula part to the base part plate at the time where it is fully inserted. The fastening means comprise outward hooks 27 that can pivot around an axe close to the body of the cannula part in such a way that the diameter formed by the outermost edge of the hooks can be reduced when the hooks are pressed inward i.e. towards the centre of the cannula part. When the pressure is removed the hooks will return to their original position due to the flexibility of the material. The hooks will be pushed inwards when they pass an opening in the base part having a cross-section which at least in one dimension is smaller than the outer edge of the hooks and as the hooks return to their original position after having passed through the opening, the hooks will lock the cannula part 7 in the inserted position.

The handling means 26 can have many other designs than the one illustrated in FIGS. 3a and 3b. The important is the function of the handling means namely that it facilitates the easy removal of a cannula part from a base part plate.

In one embodiment handling means could also be incorporated in the sensor part of a delivery device. This would ease the removal of the sensor if e.g. something is wrong with the sensor. Thereby only the sensor part needs to be removed instead of the whole patch.

FIG. 3c shows one embodiment of the connection for reservoir 6.

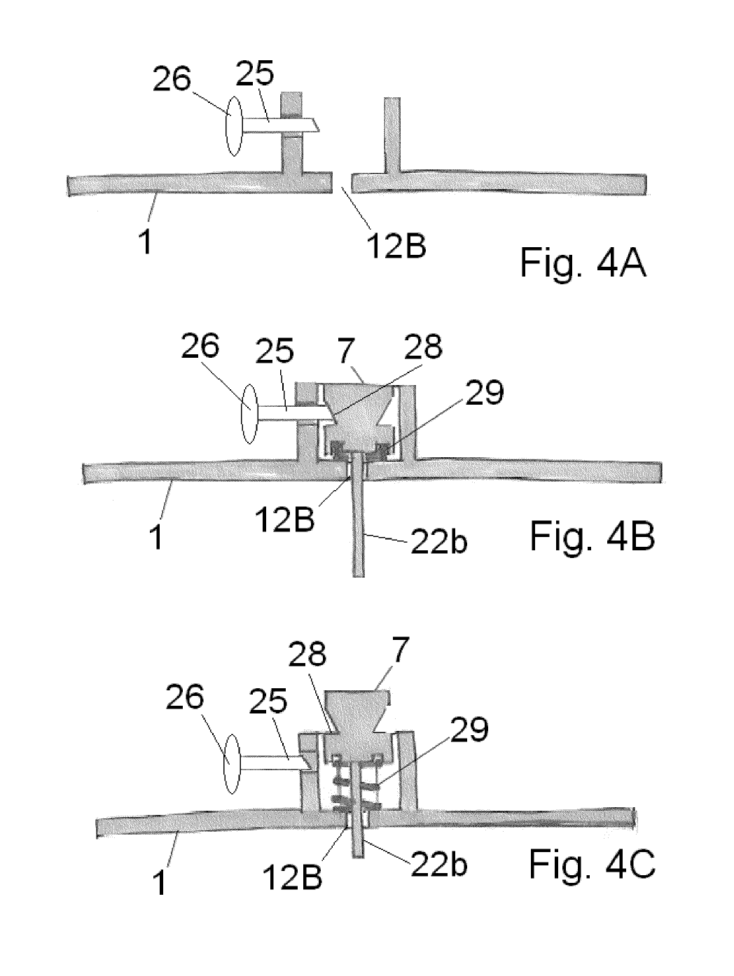

FIGS. 4A, B and C show a sideward cut-through view of a cannula part provided with fastening means 25 and illustrates how these fastenings means can lock the cannula part to the plate 1.

According to this embodiment the fastening means comprise a stick 25 or a functionally similar protruding part provided with handling means 26 which stick 25 can move relative to the plate 1 and relative to the body part 7 of the cannula part in a direction perpendicular or essentially perpendicular to the direction of insertion of the cannula. The stick 25 fits into an opening in the plate 1 and this closed opening prevents the stick 25 from moving in the insertion direction.

FIG. 4A shows a section of the plate 1 into which the cannula part is to be inserted before insertion of the cannula part. The cannula part will normally be inserted with an inserter and such an inserter might have guiding parts corresponding to the upright guiding parts 27 positioned around the opening 12B through which the cannula part is to be inserted subcutaneously. The cannula part is provided with a spring on the proximal side i.e. the side which is pushed towards the plate 1 and the patient's skin. If the inserter providing the insertion is an automatic inserter where a spring or the like is released and then forces the cannula towards the mounting position, then the force provided by the spring or the like should be large enough to force the cannula spring into a loaded position.

FIG. 4B shows the same section of the plate 1 as FIG. 4A but in FIG. 4B the cannula part has been fully inserted and is shown in a use position. Normally, the stick 25 will be elastically mounted in such a way that the position shown in FIGS. 4A and 4B is the resting position and the elastic mounting will push the stick 25 into this shown position, if it is possible.

During insertion of the cannula part, a lower surface i.e. a surface close to the patient's skin of the cannula part pushes the stick 25 outward through the closed opening when the lower surface gets into contact with the inclined surface of the end of the stick 25. As soon as the lower part of the cannula part has passed the opening in the wall of the upright guiding parts 27, the opening 28 in the body part 7 of the cannula part will allow the stick 25 to return to its resting position. When the stick 25 has been pushed into the opening 28 it is not possible for the cannula part to return back in the insertion direction although the spring 29 pushes the cannula part in this direction. When the stick 25 shoots forward to the resting position the cannula part is locked in the use position.

When the user wants to remove the cannula part, the user grasp the handling means 26 and manually pulls the stick 25 out of the opening 28 in the body part 7 whereby the cannula part can be released from the locked position in the plate 1. When the spring 29 pushes the cannula part partly out of the upright guiding parts 27, the user will be able to take hold on the body part 7 and thereby detaching the cannula part by directly grapping and pulling the used unit from the patient in a direction opposite the insertion direction. The spring 29 can either be attached to the cannula part or be a part of the base plate or be an independent unit.

Common for all the embodiments of the base part systems according to the present invention, are that the base part systems has one inlet for fluid and one or more outlets for fluid i.e. the medication enters at one position via the inlet of the first fluid path and the first fluid path is then provided with one or more outlets to one or more second fluid paths. Normally, there is no “reservoir” after the fluid has left the especially protected reservoir 6 which is used to store the fluid medication before and during use, after the fluid has left this designated reservoir 6 the fluid travels in a plug-flow assuring that all fluid has a well-defined residence time inside the base part. Generally the number of cannula sites positioned in a base part can be more than two cannula sites, the number of independent sites has no upper limitation as such but the size of the present base part plates will allow for positioning of up to around 8 independent cannula parts. This could further extend the lifetime of the device.

According to one embodiment four cannula sites could be incorporated in the device, e.g. one in each corner of the base part and in another embodiment the base part could be round whereby an unlimited number of cannula sites could be positioned around the outer sphere of the base part plate. In these embodiments it is still possible to use a movable part 90 to establish the fluid path between the reservoir and the cannula due to symmetry and by changing the position of the movable part the direction of the fluid path is changed from one cannula to another cannula.

According to one embodiment the base part itself does not have sites for cannulas but can be combined with releasable sites for a cannula. According to this embodiment the user first insert and attach the cannula part to a site, and when the user wants to detach the cannula part the user detach a releasable site together with the cannula. When a user is to start using a base part plate according to this embodiment, the base part plate is first positioned and attached to the patient's skin. Then the user places a cannula site on the patients' skin, when provided with a soft cannula the cannula site has to be positioned with an insertion needle which insertion needle might be part of a manual or an automatic inserter. The cannula connector needle is connected to the cannula site via a flexible tube through which the medication from the reservoir can flow. After approx. 3 days or if a problem such as inflammation or leakage arises with the first cannula site, the site is disconnected by retracting the cannula connector needle from the receiving portion where after the cannula site can be removed from the patients skin by grapping and pulling the handling means attached to the body part of the cannula part, the handling means allowing a user to take hold on the body part and to detach the whole cannula site with the cannula part by directly grapping and pulling the used unit from the patient in a direction opposite the insertion direction. Then a second cannula site with a soft cannula is mounted on the patient's skin and a new second fluid path is established.

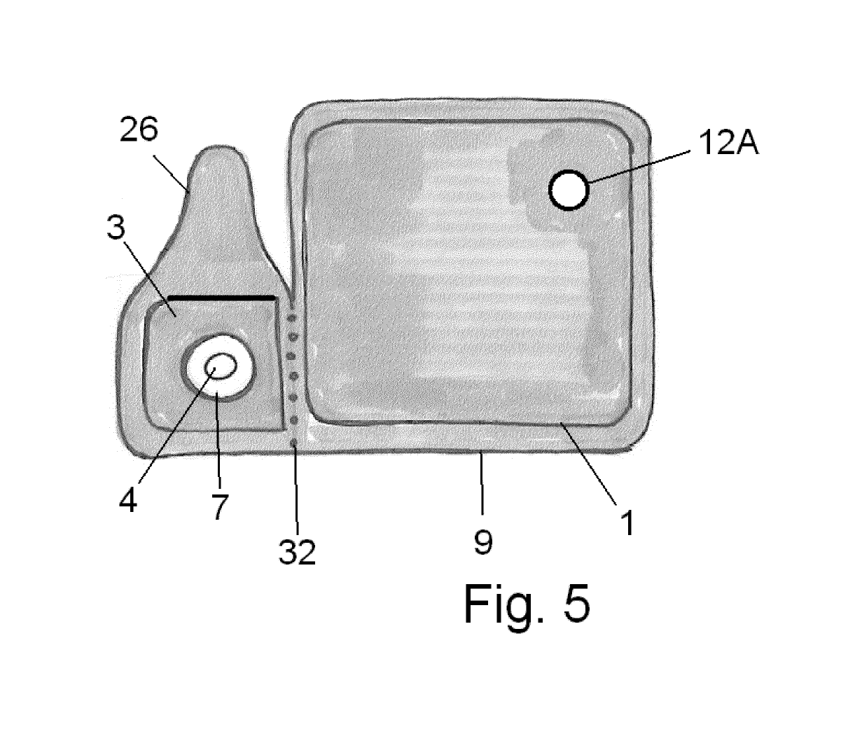

FIG. 5 shows an embodiment of a plate 1 according to the invention which plate is shown with a releasable cannula site 31. The shown embodiment comprises two rigid plates 1 and 3 which are positioned on a coherent patch 9 e.g. provided with an adhesive lower surface which can be attached to a patient's skin during use.

Alternatively, the plate can comprise a patch 9 provided with more than two relatively rigid plates 3 providing the possibility of attaching several cannula parts or sensors, or a single relatively rigid plate is provided with areas provided with lines of thin or otherwise weakened material which would make it possible for the plate 1 to bend and eventually break along such a line or to tear a part of the plate away. When the subcutaneous part of the subcutaneous unit which is attached to the removal part of the rigid plate is soft and flexible such as a soft cannula, the patient will not feel any discomfort although the subcutaneous part is not removed from the subcutaneous position in the same direction as it was inserted.

The adjacent rigid plate 3 is combined with the main plate 1 before and during attachment of the plate 1 to the user's skin, but the adjacent rigid plate 3 including the used cannula part detaches from the main plate 1 when the cannula part is removed by grapping and pulling the handling means 26. The handling means 26 according to the shown embodiment is constituted by an extension of the patch 9 which extension normally has a non-adhesive lower surface. Alternatively, the handling means could be attached to or be part of the body part 7 of the cannula part, when pulling the handling means a non-releasable attachment between the fastening means of the cannula part and the adjacent rigid plate 3 would then result in the removal of both the adjacent rigid plate 3 and the used cannula part.

According to the embodiment shown in FIG. 5, a weakened line 32 can be provided between the adjacent rigid plate 3 and the main plate by partly cutting of the patch 9 as this will ease the removal of the adjacent rigid plate 3 to which the first cannula part is attached. The number of adjacent rigid plates used with the base part could be more than two; the number of independent adjacent plates has no upper limitation as such.

1. A base part system to be combined with a detachable reservoir/delivery part, the base part system comprising:
a plate provided with a lower mounting surface and openings through which two or more subcutaneous units comprising at least two subcutaneously positioned cannulas or at least one subcutaneously positioned cannula and a subcutaneously positioned part of a sensor extend during use,
during use at least one subcutaneous unit comprising a cannula part is attached to the plate with a connector, said at least one subcutaneous unit comprises a body part placed above the patient's skin and comprising an inner cavity and an inlet opening through which fluid can enter the inner cavity and an outlet opening through which the fluid can exit the inner cavity and enter an at least partly subcutaneously positioned cannula, the body part of the at least one subcutaneous unit comprising a handle portion, said handle portion allowing a user to detach the subcutaneous unit from the plate and remove the used subcutaneous unit from the patient in a direction opposite the direction of insertion.

2. A base part system according to claim 1, wherein the plate is made of a relatively rigid material having a Young's Modulus >0.6 GPa.

3. A base part system according to claim 1, wherein the inlet opening to the inner cavity is either covered and dosed by a septum or encircled by a sealing or gasket providing a fluid tight closure to a fluid supply thereby preventing access of microorganism to the inner cavity after a cannula part has been inserted into the plate.

4. A base part system according to claim 1, wherein the handle portion not in direct contact with the connector but is positioned above the connector at a greater distance from plate than the connector.

5. A base part system according to claim 1, wherein the connector comprises a non-hardening adhesive agent allowing said subcutaneous unit to attach and to detach from the plate.

6. A base part system according to claim 1, wherein the connector comprises pivotal hooks having a locked and an unlocked position relative to the plate, allowing said subcutaneous unit to attach and to detach from the plate.

7. A base part system according to claim 1, wherein the connector is positioned at the lower side of said body part of said at least one subcutaneous unit.

8. A base part system according to claim 1, wherein a part placed subcutaneously is a soft, non-penetrating cannula.

9. A base part system according to claim 1, wherein the connector attached to said body part of said at least one subcutaneous unit comprises a surface facing the patient's skin thereby providing a contact surface making it possible for the user to affect the subcutaneous unit with a force in a direction opposite (180°) a direction in which a part placed subcutaneously is inserted into the patient and being large enough to overcome a force attaching the subcutaneous unit to the plate or the patients skin.

10. A base part system according to claim 1, wherein the connector attached to said body part of said at least one subcutaneous unit is provided with a surface positioned perpendicular to the patient's skin making it possible to first affect the subcutaneous unit with a force directed parallel to the patient's skin and being large enough to overcome a force keeping the connector of the subcutaneous unit in an attached position in the plate whereby the connector is released and second affect the subcutaneous unit with a force in a direction opposite (180°) the direction in which the part placed subcutaneously is inserted into the patient and being large enough to overcome the force attaching the subcutaneous unit to the plate or the patient's skin.

11. A subcutaneous unit comprising a body part placed above the patient's skin, a part placed subcutaneously and connector for attaching the subcutaneous unit to a base plate, the subcutaneous unit is provided with a handle portion attached to said body part, wherein the handle portion allows a user to detach the subcutaneous unit from a part of the plate and dispose of the used unit.

12. A subcutaneous unit according to claim 11, where the subcutaneously placed part of the subcutaneous unit is a soft, non-penetrating cannula.

13. A subcutaneous unit according to claim 11, wherein said handle portion comprises hooks, whereby said subcutaneous unit can be detached from a plate and removed from a patient by pressing said hooks towards each other.
